(19) United States
(12) Patent Application Publication
(10) Pub. No.: US 2025/0262154 A1
(43) Pub. Date: Aug. 21, 2025

(54) A NON-AQUEOUS SPRAY COMPOSITION

(71) Applicant: AMATERASU LIFESCIENCES LLP., Mumbai (IN)

(72) Inventor: Maharukh Rustomjee, Mumbai (IN)

(73) Assignee: AMATERASU LIFESCIENCES LLP., Mumbai (IN)

(21) Appl. No.: 19/054,605

(22) Filed: Feb. 14, 2025

(30) Foreign Application Priority Data
Feb. 16, 2024 (IN) 202421010952

(51) Int. Cl. A61K 9/12; A61K 9/00; A61K 47/34

(52) U.S. Cl. CPC A61K 9/124; A61K 9/0014; A61K 47/34

(57) ABSTRACT

A non-aqueous spray composition is provided which comprises 0.1 to 40% by weight of at least one polymeric silicone-based film-forming agent selected from dimethiconol, dimethicone/vinyl dimethicone crosspolymer, dimethicone crosspolymer, dimethicone and mixtures thereof; 10 to 60% by weight of propellant selected from compressed gas and liquified gas; and 30 to 90% by weight of silicone-based volatile solvent;
wherein % by weight is by weight of total composition. Also provided is method of forming such composition.

### FIELD OF THE INVENTION

The present invention relates to a non-aqueous spray composition and its use in preventing and treating human skin disorders.

More specifically the present invention relates to a rapid drying, non-aqueous liquid spray composition and its use in preventing and treating human skin disorders.

### BACKGROUND OF THE INVENTION

Skin when exposed to constant friction may become irritated, chaffed and could get injured or erupt as rashes (on inner thigh, under arm, under bra area, etc), groin itch, jock itch, diaper rash or bed sores. This can be a particular problem among many humans, whether caused by constant rubbing against another skin surface or by frictional exposure to an external surface such as cloth or solid surfaces depending upon the human's activity.

Common symptoms of friction related skin irritation/dermatitis and chafing is painful stinging or feeling of burning.

Chafing is associated with rash, redness, tearing or scraping of the skin exposing the skin to possible infection. Sweat and moisture play a crucial role and can aggravate friction and chafing significantly especially in hot and humid weather conditions; hence it is preferred to have a water repellent barrier so that sweat and moisture can be kept away from the skin surface and friction can be minimized.

Diaper rash is another skin condition which occurs in babies and senior citizens using diapers. In this condition the skin is sore, red, scaly, painful and tender.

Another skin condition in senior citizens is bed sores or pressure ulcers or decubitus ulcers occurring due to injuries to the skin and underlying tissue, primarily caused by prolonged pressure on the skin and shear forces (caused by friction) and local microclimate. The local microclimate relates to the temperature and humidity at the skin surface, and it is required to keep moisture away from the skin in order to reduce the occurrence of bed sores, which can be achieved with a water repellent skin protectant barrier. Moisture, sweat or urine or fecal matter in close contact with the skin for prolonged period of time (as can happen in the sacral and groin area in patients wearing diapers) can lead to softening and breakdown of skin due to maceration and lead to infection and inflammation.

Use of products to soothe skin and alleviate chafing or friction related disorders is well known, and there have been a variety of products available on the market to perform the required function such as powders, petroleum jelly, ointments, oils, lotions, creams and the like. Some of them such as ointments, lotions spread to the clothing and stain it, can block skin pores and are comedogenic and may later lose their adherence to the skin giving only short-term relief. Particulate products may not provide to the human protection from chafing due to lower adherence to the skin and may actually aggravate the skin irritation due to lumps formed on sweating and results in need for frequent application.

Hence, there is a need for a skin protectant which can cover the surface and forms a continuous film which acts as a protective barrier and reduces the impact of friction to prevent and treat erythema and frictional dermatitis. However, most of the current remedies for chafing need to be applied frequently every couple of hours to maintain and give relief from the pain and irritation on the skin. This can be very inconvenient for patient as they need to frequently go to the rest rooms and apply the topical product, hence there is a need for a product with improved adherence and abrasion resistance resulting in a longer duration of protection.

Products are available on the market that can be used to alleviate the discomfort caused due to skin disorders. These products, however, generally suffer from, among others, stickiness and greasy feel, spoiling the fabric worn by the subject.

There is still a need for a composition that can be applied (as a spray) directly to the skin, quickly, easily, and without messing one's hand with the composition, and that dries quickly following application, is non-greasy and when applied as a thin layer, is effective for preventing and treating skin disorders caused due to friction, shear and moisture.

### PRIOR ART

WO2017213505 discloses an anti-chafing spray composition comprising about 75% to about 95% propellant, about 5% to about 15% solvent, about 1% to about 10% emollient and about 0.5% to about 5% of moisture reducing agent. The solvent could be selected from alcohols, alkanes and water.

U.S. Pat. No. 8,778,406 claims a powder spray composition and method to prevent or mitigate irritation on skin of subject with a cooling sensation. The composition suggests use of about 5 to 20% by weight of hexamethyl disiloxane and/or alcohols & 30 to 90% by weight of propellant.

U.S. Pat. No. 6,949,249 prepares a diaper rash composition comprising mineral wax and mineral oil. The composition when sprayed onto the skin of a subject gives a greasy film in view of the presence of mineral oil which is not a desired property.

WO2016157112 teaches use of non-aqueous topical spray compositions of mometasone furoate with solubilizers, non-aqueous volatile solvents and film forming polymers.

Thus there is need for a composition which is anhydrous though using higher amount of solvent with lower amount of propellant rendering it to dry rapidly on application to skin of a subject and yet not a powder spray and devoid of mineral oil making film formed non-greasy and also free of solubilizers. A higher amount of solvent in a spray solution containing a film forming polymer ensures that during the process of evaporation and film formation, adequate amount of the polymer is deposited on the skin and the barrier film thus formed is uniform, hence making it useful in its application.

However the difficulty is in achieving a composition with high solvent amount and low propellant and yet allow it to be sprayed and dried fast on the site of action without dripping

### OBJECT OF THE INVENTION

An object of the present invention is to provide a non-aqueous spray composition for skin disorders.

Yet another object of the present invention is a non-aqueous spray composition which on application to skin of said subject (human) dries rapidly forming a water repellent, barrier film which is user friendly as it is non-greasy and abrasion resistant.

A further object of the present invention is to provide a spray composition which is not powder.

Another object of the present invention is to provide a spray composition with higher amount of solvent and lower amount of propellant

### SUMMARY OF THE INVENTION

A non-aqueous spray composition comprising
a. 0.1 to 40% by weight of at least one polymeric silicone-based film-forming agent selected from dimethiconol, dimethicone/vinyl dimethicone crosspolymer, dimethicone crosspolymer, dimethicone and mixtures thereof;
b. 10 to 60% by weight of propellant selected from compressed gas and liquified gas; and
c. 30 to 90% by weight of silicone-based volatile solvent;
wherein % by weight is by weight of total composition.

The non-aqueous spray composition is used to prevent or treat skin disorders selected from at least one of irritation of the skin, chafing, razor burn, itching or pruritis, scar rash, diaper rash, diaper dermatitis, athlete's foot (tinea pedis), jock itch (tinea cruris), shoe bite, skin friction, bed sores, intertrigo, ringworm, (tinea corporis) acne, candidiasis, incontinence associated dermatitis, adhesive related skin injury, Intertrigo between skin folds, friction related rashes and skin injury, pressure injury and scar as a result of a cause selected from the group consisting of scrape, cut, laceration, surgery and abrasion.

According to another aspect there is provided a method of preparing non-aqueous spray composition comprising
a. 0.1 to 40% % by weight of at least one polymeric silicone-based film-forming agent selected from dimethiconol, dimethicone/vinyl dimethicone crosspolymer, dimethicone crosspolymer, dimethicone and mixtures thereof;
b. 10 to 60% by weight of propellant selected from compressed gas and liquified gas; and
c. c. 30 to 90% by weight of silicone-based volatile solvent;
wherein said process comprises mixing film forming agent with the solvent; and finally adding the propellant.

### BRIEF DESCRIPTION OF DRAWINGS

FIG. 1: AATCC-Standard Spray Test Rating Chart

FIG. 2: Observation of Water repellency on skin

### DETAILED DESCRIPTION OF THE INVENTION

The present invention relates to a spray composition which acts as a skin protectant and prevents or treats symptoms associated with chafing, diaper rash, groin rash, jock itch, pressure ulcer or bed sores and other skin conditions caused by friction and moisture.

Film-forming sprays offer many advantages compared to conventional topical preparations such as cream, gels, lotions or ointments because they can provide uniform distribution of the film forming and other ingredients, increased functionality, lower incidence of irritation, ease of application and are more hygienic as it is hands free application and prevent any contamination. Sprays offer several advantages such as practical use, excellent coverage of the skin or wound, even distribution of the drug when applied, adjustable dosage, and spreads well. Ease of use can also increase patient and caregiver compliance.

The solvents and vehicles for applications to the skin as spray compositions overwhelmingly contain water or alcohol as solvents. Although water is volatile, it takes a very long time to dry and alcohols dry quickly but cause burning and stinging which are very uncomfortable to use. Hence there is a need to deliver to the skin protectants and other ingredients where you complete the application process in a short time such as in less than 120 secs such that it leaves a uniform and even protective film on the skin which feels smooth and non-tacky.

The present inventors have found that the disadvantages of both aqueous and alcohol based spray systems can be overcome by using non-aqueous delivery systems based on highly volatile silicone fluids (such as hexamethyldisiloxane, octamethyldisiloxane and others) as the highly volatile solvents or carriers for functional substances, protectants and other ingredients. Accordingly the present inventors have used hexamethyldisiloxane, cyclomethicone, trisiloxane, cyclopentasiloxane, cyclohexasiloxane,, octamethyldisiloxane, methyl trimethicone, ethyl methicone, capryl methicone, capryl trimethicone as solvents. Such solvent systems are found to deliver skin protective agents, anti-friction agents, skin conditioning agents, antimicrobials, anti-inflammatory agents, pain killing agents and other therapeutic agents and other materials in a fluid that spreads and evaporates quickly to give a film. Such systems can be applied by spraying without rubbing or spreading by hands which is of advantage when applying to areas such as groin area and sacral area where touching is not desired.

In another aspect, a highly viscous and tacky polymer such as a high molecular weight silicone polymer can be applied as a skin protectant against friction and moisture in a few seconds leaving the skin smooth, silky and non-tacky as contrast to highly viscous pastes and creams which are commonly used.

Compositions of the invention is advantageously applied to broken skin such as chafed skin or rashes without stinging and pain and no touching. Such spray compositions can be packed in pumpable packs with a mechanical spray pump without using a propellant, such as screw on pump on HDPE bottles or mechanical pumps crimped on aluminium can without propellant. However, spray compositions of evaporation-based film forming systems with a high amount (more than 40% w/w) of highly volatile silicone solvents such as hexamethyldisiloxane (which are 100% volatile) can generate very high vapour pressures in a closed pack spray system without a propellant; which can lead to leakage from the spray pump nozzle leading to concerns relating to transport and utility of the spray system. Further many of the volatile silicone oils are inflammable and hence any leakage from the spray container would further aggravate the risk for transportation and trade of such products.

“Spray cans” or “aerosol cans,” use propellants which are liquified gas (e.g., a chlorofluorocarbon or propane, butane, propylene, butylene, isobutane and their mixtures) having a vapor pressure at 20° C. significantly higher than atmospheric pressure.

In a typical aerosol, some of the propellant exists as a gas under pressure above the product. This gas pushes down on the liquid, forcing it up through the dip tube and out of the valve when it is opened. The propellant hence prevents leakage of the product containing highly volatile solvents by keeping it at the bottom of the can until the valve is actuated.

The propellant helps to maintain constant or near-constant pressure and creates a consistent atomized spray of the product in the form of an aerosol.

Hence, when spray compositions having a high amount of the volatile components with a smaller amount of propellant and are filled as aerosol spray systems, we gain a dual advantage of having a spray with a high solvent content resulting in uniform and consistent film on the skin and a propellant which reduced the vaporisation of the highly volatile solvent and hence prevents leakage. This makes the product risk free for transportation and trade thus make it useful for the said purpose.

The composition of the present invention dries quickly following application, leaving behind the skin protecting or treating agent or a thin film containing the desired agent, is non-greasy, water repellent and is effective for preventing and treating skin disorders. In another embodiment, this film is resistant to abrasion and stays on the skin for a long time providing long-acting effect.

The present invention is based, on the finding of novel non-aqueous spray compositions, that provide advantages during transportation and to the end user.

The present invention relates to a non-aqueous spray composition and its use to prevent or treat skin disorders.

The non-aqueous spray composition of the present invention when applied on the skin surface dries rapidly is less than 120 seconds to form a barrier film which demonstrates excellent anti-friction properties, water repellent properties and abrasion resistance.

The non-aqueous spray of the present invention imparts lubrication to skin surfaces, protects the skin surfaces from irritation, inflammation, chafing caused by friction and may assist in preventing injury to the skin surfaces, thus avoiding violation or infection of the surfaces. The non-aqueous spray of the present invention prevents aggravation of the friction related skin irritation and chafing due to presence of sweat or moisture. Further, such spray systems can be used to treat therapeutic conditions by incorporating skin conditioning and moisturising agents, antimicrobials, anti-fungal, anti-inflammatory agents, pain killing agents and other therapeutic agents and other materials in a fluid that spreads and evaporates quickly to give a film.

### Definitions

As used herein, the following terms have the meanings ascribed to them unless specified otherwise.

The term “polymeric silicone-based film forming agent” may be defined as Silicone-containing polymers or cross polymers that have ability to form films are useful herein and will typically have Silicone-seeking groups and are hydrophobic in character. Examples of silicone polymers and crosspolymers included but not limited to dimethiconol, dimethicone, dimethicone/vinyldimethicone crosspolymers, dimethicone crosspolymers, dimethicone/phenyl vinyldimethicone crosspolymers, ceteryl dimethicone/vinyldimethicone crosspolymer, vinyldimethicone/methicone silsesquioxane crosspolymers and mixtures thereof.

The term “propellant” may be defined as a gas, liquid, or solid that is used to force the contents of an aerosol out of a container. Propellants are compressed fluids or liquified gas that are released by a valve to move the contents out of the can, some aerosol cans use a compressed gas like nitrogen or carbon dioxide as their propellant. The advantage of compressed gas as an aerosol can propellant is that it's very cheap, and it isn't flammable, though other products within the aerosol can be. The disadvantage of compressed gas as an aerosol can propellant is that the pressure within the can will decrease over the life of the can.

The most common form of aerosol can propellant is a liquefied gas like propane, butane, propylene, butylene, isobutane and mixtures thereof. The advantage of this system is that it provides constant pressure levels throughout the life of the can. The disadvantage of this system is that liquid gas propellants are flammable, which increases the danger associated with their use and storage. Even so, this is still the most common form of aerosol can propellant. Chlorofluorocarbons were used for a time as an aerosol can propellant. They provided the advantages of both liquid gas and compressed gas aerosol cans, meaning that they provided constant pressure throughout the life of the can and weren't flammable. However, it was found that they were damaging the ozone layer and hence are now banned.

The term “silicone-based volatile solvent” refers to a silicone-based substance that dissolves a solute (a chemically distinct liquid, solid or gas), resulting in a solution. Liquids that evaporates readily at normal temperature (of about 25° C.) are known as volatile liquids. These are liquids that transform easily into the vapor phase. Usually, the vapor pressure of volatile liquids at 25° C. is high. A high vapor pressure usually is an indication of a volatile liquid, or one that readily vaporizes. Volatile silicones have a low heat of vaporization and a high vapour pressure and hence they evaporate quickly from the skin at ambient temperature conditions, providing a dry feel. The silicone-based volatile solvent are selected from hexamethyldisiloxane, dimethicone, cyclomethicone, trisiloxane, cyclopentasiloxane, cyclohexasiloxane, octamethyldisiloxane, methyl trimethicone, ethyl methicone, capryl methicone, capryl trimethicone and mixtures thereof.

The term “pharmaceutical excipient” refers to substance formulated alongside for the purpose of long-term stabilization, facilitating end use such as masking odors or appealing. Excipients can also be useful in the manufacturing process, to aid in film formation and in vitro stability such as prevention of decomposition or oxidation over the expected shelf life.

The term “barrier film” relates to polymeric solutions in a quick-drying solvent or silicone based spray which form a membranous cover when applied to the skin and protects skin from irritants following surgery, trauma or in chronic wounds. The barrier film is capable of reducing, relieving, or minimizing chafing from friction of human skin, particularly friction induced injury to the skin ranging from minor irritation or abrasion, resulting from the rubbing of skin against skin, clothing, shoes, or other materials. Chafing includes conditions of skin irritation and inflammation namely erythema and frictional dermatitis. Friction could be also caused in human using diapers leading to diaper rash. Friction can also cause shear on skin which can lead to bedsores in bedridden and immobile persons. Polymeric solutions can be used in hairy areas (e.g. scalp) where other types of dressings cannot stick. The barrier film can be designed to be permeable to water vapour or can be designed to prevent loss of water vapour from the skin to give occlusion if required for healing or treating skin disorders.

The term “preventing or treating” a subject includes the application of the composition described herein, to a subject with the purpose of preventing, curing, healing, alleviating, relieving, altering, remedying, ameliorating, improving, stabilizing or affecting a skin disorder, or a symptom of a skin disorder.

As used herein, the term “Abrasion resistance” means a property which allows a material to resist wearing off due to repetitive rubbing. The composition is expected to be retained on the skin for a longer time when it has a higher resistance to abrasion. This leads to a long acting protection or treatment with this type of composition which are abrasion resistant.

As used herein the term “applied on to the skin in an effective amount” means the non-aqueous spray composition being applied in an amount sufficient to cover the affected area of the skin.

The term “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as human, cat, dog, rabbit, bear, fox, wolf, monkey, deer, mouse, and pig.

As used herein the term “water repellant” refers to compositions when applied to a surface repel water or moisture and keep water away from the surface. This property as achieved by the protective film formed by the present composition ensures that the skin is protected from sweat, moisture, urine and faecal matter which can cause irritation, inflammation and rashes.

### Non-aqueous Spray Composition of the Present Invention

The present invention provides, inter alia, novel non-aqueous spray compositions and unit dosage forms containing the same. The non-aqueous spray composition is preferably liquid and effectively prevents and treats skin disorder.

The present inventors have found that high amount of silicone-based volatile solvent and low amount of propellants are key to the novel non-aqueous spray composition as it enables rapid drying of a uniform barrier film produced on application to the skin of the said subject without dripping.

As described in detail herein, the non-aqueous spray composition of the present invention comprises
a. 0.1 to 40% by weight of at least one polymeric film forming agent;
b. 10 to 60% by weight of at least one propellant; and
c. 30 to 90% by weight of at least one volatile solvent;
wherein % by weight is by weight of total composition.

More specifically, the non-aqueous spray composition of the present invention comprises
a. 0.1 to 40% by weight of at least one polymeric film forming agent selected from dimethiconol, dimethicone/vinyl dimethicone crosspolymer, dimethicone crosspolymer, dimethicone and mixtures thereof;
b. 10 to 60% by weight of propellant selected from compressed gas and liquified gas; and
c. 30 to 90% by weight of volatile solvent;
wherein % by weight is by weight of total composition.

### Polymeric Silicone-Based Film Forming Agent

The polymeric silicone-based film forming agent is selected from silicone crosspolymer(s) and may have an average molecular weight in excess of 10,000 (e.g., between about 10,000 and 10,000,000). Examples of silicone crosspolymers included but not limited to dimethicone/vinyldimethicone crosspolymers, dimethicone crosspolymers, dimethicone, dimethicone/phenyl vinyldimethicone crosspolymers, vinyldimethicone/methicone sesquioxane crosspolymers, dimethicone/PEG-10/15 crosspolymers, PEG-15/Lauryldimethicone crosspolymers and mixtures thereof. The spray composition may comprise ready-to-use silicone elastomer gels which are blends of silicone crosspolymers and silicone oils. The ready-to-use silicone elastomer gels are available with different silicone polymers dispersed and gelled in silicone oil such as cyclopentasiloxane, dimethicone, Trisiloxane and others. Typically the ready-to-use silicone gels contain around 5 to 50% by weight of the polymer. These are supplied by various manufacturers such as Shinetsu chemical company limited, DOW Silicones Corporation, Wacker Chemie AG and many others

The dimethicone when used is with viscosity of less than 1000 cps as dimethicone with higher viscosity tends to make the product sticky.

The polymeric silicone-based film forming agent may be present in 0.1 to 40% by weight of the composition; preferably 0.5 to 25% by weight of the composition and more preferably 1 to 15% by weight of the composition.

### Propellant

The propellant may be selected from compressed gas and liquefied gas.

The compressed gas may be selected from nitrogen and carbon dioxide and mixtures thereof.

The liquefied gas may be selected from propane, butane, propylene, butylene, isobutane and mixtures thereof.

The propellant may be present in 10 to 60% by weight of the composition; preferably 10 to 50% by weight of the composition and more preferably 12 to 30% by weight of the composition.

### Silicone-Based Volatile Solvent

The non-aqueous spray compositions described herein include a silicone-based volatile solvent. The silicone-based volatile solvent is selected from hexamethyldisiloxane, cyclomethicone, trisiloxane, cyclopentasiloxane, cyclohexasiloxane, octamethyldisiloxane, methyl trimethicone, ethyl methicone, capryl methicone, capryl trimethicone and mixtures thereof; preferably hexamethyldisiloxane.

Hexamethyldisiloxane is the preferred solvent as it has high vapor pressure and high volatility.

The silicone-based volatile solvent may be present in about 30 to 90 wt. % of the composition; preferably 35 to 85 wt. % of the composition and more preferably 35 to 75% by weight of the composition.

### Optional Excipients

The non-aqueous spray composition of the present invention optionally comprises 0.1 to 10% by weight of one or more excipients selected from magnesium aluminometasilicate, talc, aluminum starch octenylsuccinate, sodium starch octenylsuccinate, silica, corn starch, maize starch, tapioca starch, bentonite, kaolin, zeolite, calcium silicate, colorants, flavorants, perfumes and mixtures thereof.

The non-aqueous spray composition of the present invention may further comprise an active ingredient selected from an anti-fungal, an anti-bacterial, an anti-viral agent, an anti-inflammatory agent, wound healing agent, a pain killer or any therapeutic agent and combinations thereof.

The silicone polymer based film forming agent like Dimethiconol and silicone elastomers' like Dimethicone cross polymer in Dimethicone gel are mixed with a part of the solvent like Hexamethyldisiloxane in a closed vessel, till uniform solution was obtained. Silica silylate and other solid ingredients like starch was then dispersed in the remaining part of the solvent like Hexamethyldisiloxane and added to the solution. The other optional ingredients like oils and vitamin E, etc are added and mixed. This was mixed well to form a homogeneous solution or dispersion, which was then filled into the aerosol can followed by crimping of the valve onto the can and then filling of the pressurized liquified gas is under pressure. All operations are carried out at ambient room temperature in well closed containers.

### Packaging

The non-aqueous spray composition of the embodiments is filled in a spray can or aerosol spray dispenser according to techniques well known in the art.

In an embodiment, in order to mix the ingredients of the anti-spray composition, the solvent(s) is (are) first added. Thereafter the other non-propellant ingredients are added one by one during stirring. The resulting bulk composition is then filled into the spray can and the valve is clinched on. The propellant(s) is (are) thereafter filled into the spray can and an actuator is added.

According to another aspect, the present invention relates to a spray can or aerosol spray dispenser comprising a non-aqueous spray composition according to the embodiments.

The spray can or aerosol spray dispenser can, in various embodiments, be made of aluminium, plastic, glass or tin material in any given size and shape.

### Specific Ranges, Values and Embodiments

Specific ranges, values, and embodiments provided herein are for illustration purposes only, and do not otherwise limit the scope of the disclosed subject matter, as defined by the claims.

### Application of the Non-aqueous Spray Compositions of the Present Invention

Once formed, the non-aqueous spray composition described herein is useful for application to skin of said subject (human).

The non-aqueous spray composition of the present invention on application to skin of said subject (human) dries in less than 120 seconds.

As the composition dries very quickly following application onto the skin, the skin feels dry within couple of minutes following application of the non-aqueous spray compositions.

The prior art products take quite some time to dry following application and thereby the skin feels wet and moist.

The non-aqueous spray composition of the present invention on application to skin of said subject forms a barrier film.

The non-aqueous spray composition of the present invention on application to skin of said subject forms a barrier film for protection from friction and moisture.

The non-aqueous spray composition of the present invention on application to skin of said subject forms a barrier film wherein the film is non-greasy.

The non-aqueous spray composition of the present invention on application to skin of said subject forms a barrier film wherein the film is abrasion resistant.

The non-aqueous spray composition of the present invention on application to skin of said subject forms a barrier film wherein the film repels water.

A significant advantage of the non-aqueous spray composition of the present invention is that it is capable of providing sufficient relief even when topically applied, such as sprayed, as a very thin layer or film onto the skin. Thus, only a small amount of the non-aqueous spray composition needs to be applied onto the skin and still achieve the desired effect.

Typically, the non-aqueous spray composition is applied topically from about 1 mg/cm² to about 40 mg/cm² to the skin of the subject in a particular embodiment, from about 1 mg/cm² to about 30 mg/cm², from about 5 mg/cm² to about 30 mg/cm² to the skin of the subject.

The non-aqueous spray composition of the present invention may advantageously be used in prophylaxis applications, i.e., to prevent skin disorders.

Alternatively, or in addition, the non-aqueous spray composition may be applied to a skin area that has already been exposed to chafing. In such a case, the non-aqueous spray composition may treat the skin area by forming a protective film or layer that protects the skin from further chafing and allowing the skin area to heal. The non-aqueous spray composition may, thus, be used to inhibit, reduce or mitigate chafing in a subject.

The non-aqueous spray compositions of the embodiments achieve a desired anti-chafing and anti-friction effect even when applying a small amount and as a thin layer or film onto the skin of a subject. The non-aqueous spray compositions further have the advantage of quickly drying and presenting a non-greasy feel following application to the skin.

### Treatment of Skin Disorders

Accordingly, the non-aqueous spray composition of the present invention may be used in method of treating skin disorders selected from at least one of irritation of the skin, chafing, razor burn, itching or pruritis, scar rash, diaper rash, diaper dermatitis, athlete's foot (tinea pedis), jock itch (tinea cruris), shoe bite, skin friction, bed sores, diabetic foot ulcer, venous ulcers, intertrigo, ringworm, (tinea corporis) acne, candidiasis, incontinence associated dermatitis, adhesive related skin injury, Intertrigo between skin folds, friction related rashes and skin injury, pressure injury and scar as a result of a cause selected from the group consisting of scrape, cut, laceration, surgery and abrasion.

The method of treatment of such skin orders, comprises applying topically the non-aqueous spray composition of the present invention from about 1 mg/cm² to about 40 mg/cm² to the skin of the subject in a particular embodiment, from about 1 mg/cm² to about 30 mg/cm², from about 5 mg/cm² to about 30 mg/cm² to the skin of the subject in need thereof.

The following examples illustrate preferred embodiments in accordance with the present invention without limiting the scope of the invention.

### EXAMPLES

### Example 1

### Non-Aqueous Spray Compositions Without Propellant

| | Objective | | |
|---|---|---|---|
| | Batch with high silicone solvent HMDSO | | |
| | Example No | | |
| | 1.1 | 1.2 | 1.3 |
| | % w/w | % w/w | % w/w |
| Dimethicone Cross | 0 | 10 | 1 |
| polymer in dimethicone | | | |
| gel | | | |
| Silica Silylate | | 0.5 | 0.7 |
| Dimethiconol | 5 | 3 | 5 |
| Hexamethyldisiloxane | 93 | 77.2 | 84 |
| Dimethicone 350cps | 2 | 2 | 2 |
| maize starch | 0 | 7 | 7 |
| Lemongrass oil | 0 | 0.3 | 0.3 |
| Total | 100.00 | 100.00 | 100.00 |
| Spray pumpable | Yes | Yes | Yes |
| Drying time on skin | Yes | Yes | Yes |
| in <120 seconds | | | |
| Pack | HDPE bottle with | HDPE bottle with | aluminium can |
| | screwed on | screwed on | with a crimped |
| | mechanical pump | mechanical pump, | on mechanical |
| | | further packed in | pump without |
| | | sealed PET/poly | propellant |
| | | pouches. | |
| Leakage/Non leakage | Loosening and | Ballooning of | Leakage from |
| | unscrewing of | pouch and | mechanical |
| | cap and Leakage | Loosening and | pump. |
| | | unscrewing of cap | |
| | | and Leakage | |
| | | Leakage | |

Process: The Dimethiconol and Dimethicone cross polymer in Dimethicone gel were mixed with part of the Hexamethyldisiloxane is a closed vessel, till uniform solution was obtained. Silica silylate was then dispersed in the remaining part of Hexamethyldisiloxane and added to the solution. This was mixed well to form a homogeneous dispersion, which was then filled into the packs.

Example 1.1 and 1.2 were filled in HDPE bottles with screw on HPDE pump caps and tightened well.

The formulation of example 1.1 was packed in HDPE container with tightened screw cap and placed for storage testing at 45° C. On storage at 45° C. for a month it was observed that the screw cap loosens and is half unscrewed. This caused leakage and is not acceptable for commercialization of the product as it would cause leakage during transportation, storage and use at customer end in addition to a safety risk due to known flammability of HMDSO.

For formulation of example 1.2, the product was packed in screwed-on HDPE pump spray bottles like example 1.1 and then further each bottle was packed in sealed PET/poly pouches, after storage of 1 month at 45° C. it was observed that the PET/poly pouches were ballooned and screw caps were loosened leading to leakage. This may be due to high vapor pressure of HMDSO and insufficient headspace (30% v/v).

The formulation of example 1.3 was packed in crimped on mechanical pump on an Aluminium canister with more than 50% v/v of headspace to take care of leakage. However, this example also showed significant leakage. After transportation and storage at ambient temperature for 3 months, more that 40% of canisters were found to leak.

Leakage Test for non-pressurized spray containers: The test to identify leakage from the crimped canisters was developed using an orbital shaker:

The canisters were kept in horizontal or vertical position in an orbital shaker at temperature of 50° C. for 4 hours. Record the observation of each canister by opening the cap and observing liquid leaked inside the cap from the pump actuator.

One of the notable characteristics of hexadimethylsiloxane is its relatively high vapor pressure. Vapor pressure refers to the pressure exerted by a vapor when it is in equilibrium with its liquid or solid form. In the case of hexadimethylsiloxane, its high vapor pressure means that it readily evaporates at room temperature and atmospheric pressure. This vapour pressure if not suppressed makes the product unstable in non-pressurised aerosol containers.

### Example 2

Compositions of the Current Invention (Non-Aqueous Spray Composition with Propellant)

| | Example No | | | | | |
|---|---|---|---|---|---|---|
| | 2.1 | 2.2 | 2.3 | 2.4 | 2.5 | 2.6 |
| Ingredients | % w/w | % w/w | % w/w | % w/w | % w/w | % w/w |
| Dimethicone | 3.9 | 3.0 | 2.5 | 2.5 | 3.9 | 3.9 |
| Crosspolymer gel in | | | | | | |
| dimethicone | | | | | | |
| Silica Silylate | 0.5 | 0.4 | 0.4 | 0.4 | 0.5 | 0.2 |
| Dimethiconol | 3.9 | 3.0 | 2.5 | 2.5 | 3.9 | 3.9 |
| Hexamethyldisiloxane | 61.6 | 47.2 | 40.5 | 43.4 | 63.1 | 67.4 |
| Dimethicone/ | 0.0 | 0.0 | 0.0 | 0.0 | 0.0 | 0.0 |
| vinyl dimethicone | | | | | | |
| crosspolymer gel in | | | | | | |
| dimethicone | | | | | | |
| Dimethicone 350 cps | 1.5 | 1.2 | 4.0 | 1.0 | 1.5 | 1.5 |
| Cyclopentasiloxane | 0.0 | 0.0 | 0.0 | 0.0 | 3.9 | 0.0 |
| maize starch | 5.4 | 4.1 | 0.0 | 0.0 | 0.0 | 0.0 |
| Lemongrass oil | 0.2 | 0.2 | 0.2 | 0.2 | 0.2 | 0.2 |
| LPG | 23.0 | 41.0 | 50.0 | 50.0 | 23.0 | 23.0 |
| Total | 100.0 | 100.0 | 100.0 | 100.0 | 100.0 | 100.0 |
| Drying time on | Yes | Yes | Yes | Yes | Yes | Yes |
| skin <120 seconds | | | | | | |
| Leakage testing | NO | NO | NO | NO | NO | NO |
| | leakage | leakage | leakage | leakage | leakage | leakage |

Process: The Dimethiconol and/or Dimethicone cross polymer in Dimethicone gel/Dimethicone/vinyldimethicone crosspolymer in dimethicone or dimethicone were mixed with part of the Hexamethyldisiloxane is a closed vessel till uniform solution was obtained. Silica silylate and starch were then dispersed in the remaining part of Hexamethyldisiloxane and added to the solution. The other ingredients were added, and this bulk solution was mixed well to form a homogeneous dispersion.

The bulk solution was then filled into aluminium cans, closed and sealed with an aerosol valve (which can withstand pressure) by crimping. The propellant fluid (liquified gas) is then pumped into the container under pressure via the valve system.

The filled pressurised aerosol containers are checked online for leakage as given below
I. Set the water bath at 53° C. and keep the water level such as the cans are dipped completely in the water bath.
II. Remove the actuator of aerosol cans and dip the cans into the water bath at 53° C.
III. Keep the cans in water bath for 3 minutes.
IV. Observe the Cans for formation of bubbles. No bubbles suggest no leakage.

The composition from examples were tested for their Drying time on skin as given below

### Procedure

1. A group of 4 panellists were chosen.
2. The forearm of the panelists were chosen as the test area. It was made sure the skin is clean, dry, and free of any lotions or other products.
3. The spray formulation was thoroughly shaken, holding the spray bottle 6 cm away from the skin, spraying the sample formulation for 1 second to make sure a uniform amount is applied to the designated area.
4. the start time was noted immediately upon spraying.
5. the spray area was observed until it no longer appears wet or shiny, using a timer to record the drying time.
6. Also, dryness was confirmed by lightly touching with a clean fingertip without disrupting the formulation film.

A drying time of less than 120 seconds on the skin ensures a smooth, continuous and uniform barrier film while preventing drips and smudges. It allows for quicker application, reduces the risk of contamination, and improves efficiency, making it a convenient option for both industrial and consumer use.

### Example 2 (continued)

### Compositions Of The Current Invention

| | | Example No | | | | | | | | |
|---|---|---|---|---|---|---|---|---|---|---|
| | 2.7 | 2.8 | 2.9 | 2.10 | 2.11 | 2.12 | 2.13 | 2.14 | 2.15 | 2.16 |
| Ingredients | % w/w | % w/w | % w/w | % w/w | % w/w | % w/w | % w/w | % w/w | % w/w | % w/w |
| Dimethicone | 10.0 | 15.4 | 0.0 | 0.0 | 0.0 | 0.0 | 3.9 | 7.7 | 0.0 | 0.0 |
| Crosspolymer | | | | | | | | | | |
| gel in | | | | | | | | | | |
| dimethicone | | | | | | | | | | |
| Silica | 0.2 | 0.2 | 0.2 | 0.2 | 0.2 | 0.2 | 0.5 | 0.4 | 0.0 | 0.43 |
| Silylate | | | | | | | | | | |
| Dimethiconol | 0.0 | 0.0 | 15.4 | 7.7 | 0.0 | 0.0 | 3.9 | 2.3 | 4.3 | 6.0 |
| Hexamethyl | 65.1 | 59.7 | 59.7 | 67.4 | 65.1 | 72.8 | 60.8 | 59.4 | 80.8 | 70.7 |
| disiloxane | | | | | | | | | | |
| Dimethicone/ | 0.0 | 0.0 | 0.0 | 0.0 | 10.0 | 2.3 | 0.0 | 0.0 | 0.0 | 0.0 |
| vinyl | | | | | | | | | | |
| dimethicone | | | | | | | | | | |
| crosspolymer | | | | | | | | | | |
| gel in | | | | | | | | | | |
| dimethicone | | | | | | | | | | |
| Dimethicone | 1.5 | 1.5 | 1.5 | 1.5 | 1.5 | 1.5 | 7.7 | 1.5 | 0.0 | 1.7 |
| 350 cps | | | | | | | | | | |
| Cyclopentasiloxane | 0.0 | 0.0 | 0.0 | 0.0 | 0.0 | 0.0 | 0.0 | 0.0 | 0.0 | 0.0 |
| maize starch | 0.0 | 0.0 | 0.0 | 0.0 | 0.0 | 0.0 | 0.0 | 5.4 | 0.0 | 6.0 |
| Lemongrass | 0.2 | 0.2 | 0.2 | 0.2 | 0.2 | 0.2 | 0.2 | 0.2 | 0.0 | 0.3 |
| oil | | | | | | | | | | |
| LPG | 23.0 | 23.0 | 23.0 | 23.0 | 23.0 | 23.0 | 23.0 | 23.0 | 15.0 | 15.0 |
| Total | 100.0 | 100.0 | 100.0 | 100.0 | 100.0 | 100.0 | 100.0 | 100.0 | 100.0 | 100.0 |
| Drying time | Yes | Yes | No | No | Yes | Yes | Yes | Yes | Yes | Yes |
| on skin <120 | | | (>300) | (>150) | | | | | | |
| seconds | | | | | | | | | | |
| Leakage | NO | NO | NO | NO | NO | NO | NO | NO | NO | NO |
| testing | leakage | leakage | leakage | leakage | leakage | leakage | leakage | leakage | leakage | leakage |

Examples 2.7 to 2.16 are prepared and tested for leakage as given in examples 2.1 to 2.6 and drying time on skin.

When stored in containers, particularly spray containers or non-pressurised spray cans, the high vapor pressure of hexadimethylsiloxane can lead to issues such as leakage. If the container is not properly sealed or if it is subjected to fluctuations in temperature or pressure, the vapor pressure of the hexadimethylsiloxane can cause it to escape from the container, resulting in leakage.

This leakage can be problematic, especially in applications where precise dosing or containment of the substance is necessary. It can also lead to wastage of the product and potential safety hazards if the leaked substance comes into contact with incompatible materials or surfaces.

To mitigate issues related to leakage in spray containers or other storage vessels, it is important to ensure proper sealing and storage conditions. Therefore, developmental trials with propellant containing (Aerosol type) formulations were conducted. The gaseous propellant keep the HMDSO which is a liquid form at the bottom of the container because of lower density.

When propellants are added to aerosol formulations containing HMDSO, they can help reduce the overall vapor pressure of the mixture. This reduction in vapor pressure can occur because it is effectively diluting the concentration of HMDSO vapor in the headspace of the container.

By reducing the vapor pressure of HMDSO, propellants can help minimize issues such as leakage from spray containers or aerosol cans. Additionally, propellants play a crucial role in dispensing the product in a controlled manner when the aerosol container is activated, ensuring that the desired amount of HMDSO is released. Therefore, the ratio of Solvent to Propellant is important in the formulation.

Further it is observed from above results that the composition according to example 2.9 and 2.10 which do not have the dimethicone cross polymer but has dimethiconol has a drying time which is more than 120 seconds while there is no leakage, whereas the other compositions which are according to the present invention have desirable drying time of less than 120 seconds. This shows the criticality of the components of the present invention in achieving the end result. Accordingly these would be outside the purview of this invention.

However examples 2.15 and 2.16 which does not have the dimethicone cross polymer but has dimethiconol (in lower amount) has a drying time which is less than 120 seconds and is considered within the purview of this invention

### Example 3

### Evaluation of the Spray Products in Example 2 (2.3, 2.6, 2.10, 2.13, 2.15) was Undertaken for Various Functional Parameters

### 3.1 Coefficient of Friction

The coefficient of friction of the above examples was tested to understand the ability of the composition to reduce the friction on the surface on the substrate when applied. The coefficient of friction was tested as per ASTM D1894 using parameters and procedure as listed below
Instrument Name: Coefficient of friction tester
Model/Product No.: MXD-02
Method: ASTM D 1894

### Procedure

1. Prepare the sample by using polyester fabric as the base and evenly spreading 1 gram of the material over a 6.3 cm×6.3 cm area.
2. Dry the sample in a dry heat oven at 90° C. for 3 hours.
3. Use a 200 g sled to slide across the surface of the sample, with a machine measuring the force required to move the sled at a constant speed of 150 mm/min.
4. Begin the test by attaching the prepared sample to the sled and operating the machine.
5. Measure two parameters: the force required to initiate movement (static friction) and the force required to maintain movement (kinetic friction).
6. Conduct the test using untreated fabric on a stainless steel (SS) surface as a control and then test the coated sample on the SS surface.

| Sr. No | Example | Coefficient of friction | % reduction in friction |
|---|---|---|---|
| 1. | Example 2.3 | 0.371 | 62.9 |
| 2. | Example 2.6 | 0.001 | 99.9 |
| 3. | Example 2.10 | 0.214 | 94.3 |
| 4. | Example 2.13 | 0.539 | 61.8 |
| 5. | Example 2.15 | −0.032 | 100 |

The above results confirm that the surface of the substrate after application of the compositions of the inventions demonstrate the ability to reduce the friction of the surface by more than 50%.

### 3.2 Water Repellency Test

The Water repellency of the above examples was tested as per ASTM standard and on skin to ensure the formed layer repels the water and hence protects the skin from maceration due to moisture or sweat.

The water repellency was tested as per below parameters and procedures

### 3.2.1. Water Repellency as per ASTM Method

Instrument Name: Spray Rating Tester

Make: SDL

Method: AATCC 22 (American Association of Textile Chemists and Colorists)

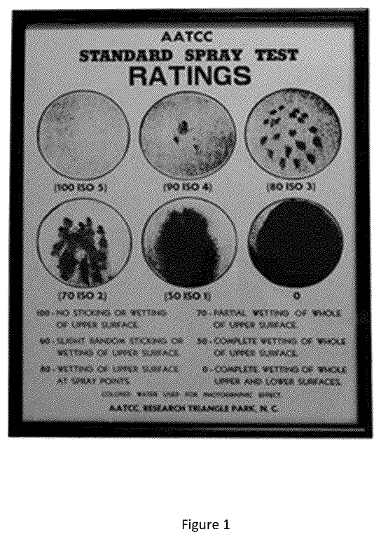

Spray Test for water Repellency. provided in FIG. 1

### Procedure

Apply 2.77 g of the sample to a 176.71 cm² polyester fabric and allow it to saturate at room temperature for 30 minutes. Secure the fabric in a metal hoop, ensuring a smooth surface, and place it face-up on the tester. Spray 250 ml of distilled water onto the fabric for 25-30 seconds, with the spray centered on the fabric. After removing the hoop, check for water penetration on the back of the fabric. Tap the fabric lightly on both sides to remove excess water. Finally, compare the wetting of the fabric to a photographic standard spray test rating chart (as shown in FIG. 1) and grade accordingly.

| Sr. No | Example | Water repellency |
|---|---|---|
| 1. | Example 2.1 | 70 |
| 2. | Example 2.2 | 70 |
| 3. | Example 2.3 | 80 |
| 4. | Example 2.5 | 80 |
| 5. | Example 2.6 | 80 |
| 6. | Example 2.8 | 90 |
| 7. | Example 2.9 | 80 |
| 8. | Example 2.10 | 80 |
| 9. | Example 2.11 | 90 |
| 10. | Example 2.13 | 90 |
| 11. | Example 2.14 | 40 |
| 12. | Example 2.16 | 70 |

The above examples form a layer on skin which does not allow water or sweat to stay on the skin. It sticks to the skin and repels the water and sweat. Sweat and moisture aggravate chafing, and formation of bed sores and incontinence associated dermatitis. tested as per the procedure below.

### Procedure

1. 5 boxes are drawn, each measuring 5×5 cm, on the volar side of the arms of the panelist.
2. a 1% amaranth dye solution is prepared and applied it to the boxes, the dye is allowed to dry completely before applying a second layer.
3. Once the dye is fully dried, calculation and spraying of the required amount is carried out to achieve 6.025 mg/cm² of solid content onto the skin.
4. It is ensured that no spray is applied to the blank area.
5. the spray is allowed to dry completely for 10 minutes
6. water is sprayed onto the dried film, applying approximately 4 short sprays to fully wet each box.
7. After a few seconds, the water is gently dabbed with a tissue to remove excess moisture.
8. the spraying and dabbing process repeated 3 times, then the color intensity of the dye is compared to the initial state.

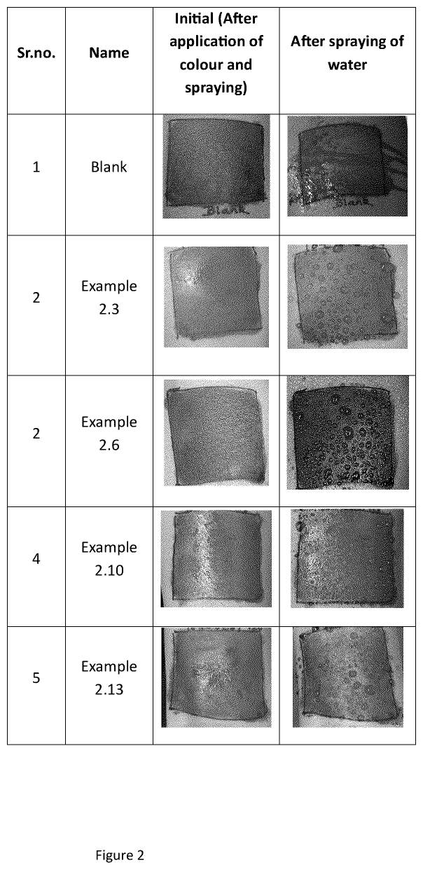

The results of water repellency is provided in the FIG. 2.

It is observed that:

For blank: The colour from the skin in the blank arm (without any protective barrier) spray is completely removed after 2^{nd} wash

For Example 2.3: Water droplets were observed on the barrier film after spraying unlike the blank due to its repellency to water. The colour intensity of the product remains same as initial upto three washes indicating its water repellency.

For Example 2.6: Water droplets were observed on the barrier film after spraying unlike the blank due to its repellency to water. The colour intensity of the product remains same as initial upto three washes indicating its water repellency.

For Example 2.10: Water droplets were observed on the barrier film after spraying unlike the blank due to its repellency to water. The colour intensity of the product remains same as initial upto three washes indicating its water repellency

For example 2.13; Water droplets were observed on the barrier film after spraying unlike the blank due to its repellency to water. The colour intensity of the product remains same as initial upto three washes indicating its water repellency.

The observation of water droplets on the product after spraying, along with the retention of color intensity for up to three washes, suggests that the barrier film of the spray formulations exhibits effective water repellency and durability on human skin. The control without any barrier film is washed off in two washes. Example 2.10 showed good water repellency and durability on human skin even though it did not show rapid drying on the skin and had a sticky feel.

Hence this shows the criticality of the ingredients and the composition for it to show the required attributes such as drying time on the skin below 120 secs, effective water repellency and durability on human skin. Accordingly formulations having all these attributes are considered within the purview of the present invention while formulations like those of Examples 2.9, 2.10, 2.14 missing at least one attribute are considered beyond invention.

The examples were also tested for resistance to accelerated abrasion as per ASTM standard method and abrasion on skin to help understand the strength of the protective film to withstand rubbing off and give a longer duration of protection. The abrasion resistance test was conducted using the following equipment and parameters.

### 3.3 Abrasion Resistance/Resistance to Rub Off

The ability of the barrier film of the spray formulation to stick to the skin and resist any rub off due to abrasion is important to ensure long time protection to the skin.

### 3.3.1. Abrasion Resistance as per ASTM Method

Standard Test Method No. customized based on ASTM D 3885/IS 12673-1989

Type of Abradant used: Zero Emery Paper

Type of Abrasion: Unidirectional

Instrument Name: Universal Wear Tester

Model/Product No.: M282

Make: SDL ATLAS, HONG KONG
Air Pressure Used: 4 psi
Load Used: 0.5 lb.
Mode: Unidirectional
Stroke Length: 1 inch

### Procedure

1. 1 g of spray is weighed, into that added 0.01 g of Isotane red Dystar. Mixed it well.
2. After mixing the sample is sprayed on the substrate (Polyester fabric) having area of 20.25 sq cm (diameter 9 cm).
3. the substrate is kept at 90° C. dry heat oven in analytical lab for 3 hours and after 3 hours the samples are transferred into a desiccator containing silica gel.
4. the dried substrate is put on the machine (SDL Atlas M282 universal wear tester). the machine is set at Uni direction and Air pressure regulator at standard 4 psi.
5. zero emery paper is used to see the abrasion resistance.
6. the machine is started and the cycle's readings are noted down when abrasion of peeling off of the film is observed
7. the substrate is visually observed when abrasion is seen.

| | | Observation (No of cycles to peel |
|---|---|---|
| Sr. No | Example | off) |
| 1. | Example 2.1 | 30 |
| 2. | Example 2.2 | 30 |
| 3. | Example 2.3 | 30 |
| 4. | Example 2.5 | 30 |
| 5. | Example 2.6 | 50 |
| 6. | Example 2.10 | 50 |
| 7. | Example 2.11 | 10 |
| 8. | Example 2.13 | 50 |
| 9. | Example 2.15 | 30 |

The above results confirm that the surface of the substrate, after application of the compositions of the invention, forms a strong film. When tested for forced abrasion, it shows a longer residence time of above 10 cycles, providing the long-lasting protection and anti-friction effect. However, the values range between 10 to 50 cycles for various examples, once again demonstrating the criticality of the ingredients in the compositions.

### 3.3.2. Abrasion Resistance on Skin

The abrasion resistance test was conducted using the following procedure on human of one volunteer.
1. 5×5 cm box is drawn on the volar side of the arm.
2. 443 mg of each sample is weighed and a pinch of BRM orange powder is added to it. Mixed thoroughly with finger.
3. the prepared mixture is applied onto the marked area on the volar side of the arm.
4. a hair dryer is used to dry the applied sample.
5. Once the sample is fully dried, an electric massager is used to rub off the film.
6. the massager is placed on the dried film and rubbed in a clockwise motion to determine the number of cycles needed to completely remove the film.
7. the cycle count is recorded when the film is entirely removed.

| | | No. of cycles required for |
|---|---|---|
| Sr no. | Product | complete removal |
| 1 | Example 2.3 | 110 |
| 2 | Example 2.6 | 100 |
| 3 | Example 2.10 | 125 |
| 4 | Example 2.13 | 112 |

Example 2.3 took 110 cycles, Example 2.6 took 100 cycles, Example 2.10 took 125 cycles, and Example 2.13 took 112 cycles. This shows that most of the samples had similar durability. All the samples were fairly resistant to being rubbed off, needing more than 50 cycles to come off completely. This demonstrates that the film formed is very flexible and strong enough to avoid being rubbed off easily. Thus, ensuring long time protection of the skin.

### 3.4 Retention on the Skin

The Retention on skin of human volunteers of the above examples was tested to ensures the product stays effective for a longer time after application. This allows the active ingredients, like those offering protection, to work as intended. The retention on skin was tested as per below procedure
1. 5×5 cm Boxes are drawn on the volar side of the arms of 4 panellists
2. the required amount of product is sprayed to achieve 6.025 mg/cm² of solid content onto the skin.
3. the product is allowed to stay on the skin for 8 hours.
4. After 8 hours, the film is visually checked and the area gently touched to see if it is still present. Observed whether the product feels the same, or if it has become dry, sticky, or greasy.

| | | Retention on skin after 8 |
|---|---|---|
| Sr. No | Example | hours |
| 1. | Example 2.1 | Yes (slippery feel) |
| 2. | Example 2.2 | Yes (slippery feel) |
| 3. | Example 2.3 | Yes (slippery feel) |
| 4. | Example 2.4 | Yes (slippery feel) |
| 5. | Example 2.5 | Yes (slippery feel) |
| 6. | Example 2.6 | Yes (slippery feel) |
| 7. | Example 2.7 | Yes (slippery feel) |
| 8. | Example 2.8 | Yes (slippery feel) |
| 9. | Example 2.9 | Yes (sticky and shiny feel) |
| 10. | Example 2.10 | Yes (sticky and shiny feel) |
| 11. | Example 2.11 | Yes (slippery feel) |
| 12. | Example 2.12 | Yes (slippery feel) |
| 13. | Example 2.13 | Yes (slippery feel) |
| 14. | Example 2.14 | Yes (slippery feel) |

The above examples of the retention test showed that the product remains effectively on the skin for the duration of 8 hours, maintaining its intended feel without becoming dry, sticky, or greasy, except for examples 2.9 and 2.10 which showed a sticky feel and also had a longer drying time as seen earlier. This confirms that the spray provides long-lasting performance and stays in place, offering the desired protection or benefits throughout its application period.

4.7 The Storage testing/leakage testing of the above examples was tested ensuring the safety and reliability of aerosol cans during storage, particularly in environments with varying temperatures, this test helps verify that the cans maintain their seal and do not leak, which could lead to product failure or safety hazards. The results justify that the cans meet the necessary standards for long-term storage and use. The Storage testing was tested as per procedure given above.

Packs of example 2.6 were studied for stability at accelerated and real time storage conditions of 40° C./75% RH and 30° C./75% RH for more than 12 months. The packs were removed periodically every 3 months and tested for physical and chemical test parameters and leakage as well as weight loss which may happen due to loss of gas/vapour from the can.

The samples were complying to all the physical and chemical test parameters as per the limits.

Leakage was not observed in any of the packs upto 12 months

Weight loss observed was well within the accepted pharmacopeial limit of less than 5% over this time period.

Hence based on this it is confirmed that the product has a shelf life of 24 months. With this proven stability the product can be commercialized in trade and through traditional supply chains.

1. A non-aqueous spray composition comprising:
a. 0.1 to 40% % by weight of at least one polymeric silicone-based film-forming agent selected from dimethiconol, dimethicone/vinyl dimethicone crosspolymer, dimethicone crosspolymer, dimethicone and mixtures thereof;
b. 10 to 60% by weight of propellant selected from compressed gas, liquefied gas and mixture thereof; and
c. 30 to 90% by weight of silicone-based volatile solvent;
wherein % by weight is by weight of total composition.

2. The composition as claimed in claim 1, wherein the silicone-based volatile solvent is selected from hexamethyldisiloxane, cyclomethicone, trisiloxane, cyclopentasiloxane, cyclohexasiloxane, octamethyldisiloxane, methyl trimethicone, ethyl methicone, capryl methicone, capryl trimethicone and mixtures thereof.

3. The composition as claimed in claim 1, wherein the polymeric silicone-based film forming agent is present in amount of 0.5 to 25% by weight.

4. The composition as claimed in claim 1, wherein propellant is present in amount of 10 to 50% by weight.

5. The composition as claimed in claim 1, wherein the compressed gas as propellant is selected from nitrogen and carbon dioxide and mixtures thereof.

6. The composition as claimed in claim 1, wherein the liquefied gas as propellant is selected from propane, butane, propylene, butylene, isobutane and mixtures thereof.

7. The composition as claimed in claim 1, further comprising an active ingredient selected from an anti-fungal, an anti-bacterial, an anti-viral agent, anti-inflammatory agents, pain killing agents and other therapeutic agents and combinations thereof.

8. The composition as claimed in claim 1, wherein the composition dries in less than 120 seconds on application to skin.

9. The composition as claimed in claim 1, wherein the composition forms a barrier film on application to skin.

10. The composition as claimed in claim 9, wherein the barrier film formed on application to skin provides protection from friction and moisture.

11. The composition as claimed in claim 10, wherein the film is non-greasy.

12. The composition as claimed claim 10, wherein the film is abrasion resistant.

13. The composition as claimed in claim 10, wherein the film repels water.

14. The composition as claimed in claim 1, when applied to skin forms a barrier film adapted in preventing or treating skin disorders selected from at least one of irritation of the skin, chafing, razor burn, itching or pruritis, scar rash, diaper rash, diaper dermatitis, athlete's foot (tinea pedis), jock itch (tinea cruris), shoe bite, skin friction, bed sores, intertrigo, ringworm, (tinea corporis) acne, candidiasis, incontinence associated dermatitis, adhesive related skin injury, Intertrigo between skin folds, friction related rashes and skin injury, pressure injury and scar as a result of a cause selected from the group consisting of scrape, cut, laceration, surgery and abrasion.

15. The composition as claimed in claim 1, wherein the polymeric silicone-based film forming agent is present in amount of 1 to 15% by weight.

16. The composition as claimed in claim 1, wherein propellant is present in amount of 12 to 30% by weight.

17. A method of preparing non-aqueous spray composition comprising
a. 0.1 to 40% % by weight of at least one polymeric silicone-based film-forming agent selected from dimethiconol, dimethicone/vinyl dimethicone crosspolymer, dimethicone crosspolymer, dimethicone and mixtures thereof;
b. 10 to 60% by weight of propellant selected from compressed gas and liquified gas; and
c. c. 30 to 90% by weight of silicone-based volatile solvent;
wherein said process comprises mixing film forming agent with the solvent; and finally adding the propellant.

18. A method of treating skin disorders selected from at least one of irritation of the skin, chafing, razor burn, itching or pruritis, scar rash, diaper rash, diaper dermatitis, athlete's foot (tinea pedis), jock itch (tinea cruris), shoe bite, skin friction, bed sores, diabetic foot ulcer, venous ulcers, intertrigo, ringworm, (tinea corporis) acne, candidiasis, incontinence associated dermatitis, adhesive related skin injury, Intertrigo between skin folds, friction related rashes and skin injury, pressure injury and scar as a result of a cause selected from the group consisting of scrape, cut, laceration, surgery and abrasion;
said method comprising applying topically from about 1 mg/cm² to about 40 mg/cm² to the skin of the subject in need thereof.
